(19) United States
(12) Patent Application Publication
(10) Pub. No.: US 2025/0057750 A1
(43) Pub. Date: Feb. 20, 2025

(54) COSMETIC COMPOSITION

(71) Applicant: GIVAUDAN SA, VERNIER (CH)

(72) Inventors: Daniel AURIOL, Toulouse (FR); Morgane DE TOLLENAERE, Pomacle (FR); Romain REYNAUD, Toulouse (FR); Amandine SCANDOLERA, Pomacle (FR)

(21) Appl. No.: 18/726,033

(22) PCT Filed: Jan. 24, 2023
(86) PCT No.: PCT/EP2023/051641

§ 371 Date: Jul. 1, 2024

(30) Foreign Application Priority Data
Jan. 24, 2022 (GB) 2200880.9

(51) Int. Cl. A61K 8/60; A61K 8/19; A61K 8/365; A61K 8/44; A61K 8/49; A61Q 19/00

(52) U.S. Cl. CPC A61K 8/60; A61K 8/19; A61K 8/365; A61K 8/44; A61K 8/4913; A61Q 19/00

(57) ABSTRACT

Cosmetic active agents and methods that are useful to reduce the number and/or size of pores in human skin are provided.

### COSMETIC COMPOSITION

The invention relates to cosmetic active agents and methods that are useful to reduce the number and/or size of pores in human skin.

The desire to appear attractive is naturally rooted in modern consumers. Even as the ideal of attractiveness undergoes change over the course of time, it is universally accepted that the condition and appearance of our skin is a significant contributor to an attractive outward appearance.

Today's consumers are offered a multitude of cosmetic products for the care of skin. Generally, these products are in the form of creams and lotions, containing water for moisturizing the skin, and fats and lipids for re-greasing it, and their effects are exerted on the outer-most layer of the skin.

Pores are small openings on the skin that sweat and sebum pass through to reach the skin's surface. A pore is the opening of a hair follicle. The sebaceous gland within each hair follicle secretes sebum, a lubricating oil, through the pores. Sebum production is important for skin health because it protects and moisturizes the skin. Facial pores are typically visible to the naked eye and can range from approximately 250 to 500 micrometers in diameter. The usual size range varies depending on factors such as skin tone and age. Enlarged pores are those which appear dilated and are clearly visible to the naked eye.

Pore size is primarily genetic, but overproduction of sebum, or oil, can also lead to visibly enlarged pores due to oil combining with skin debris, which causes clogging. Skin aging and low skin elasticity may also make pores appear enlarged. Other factors include chronic acne, hormonal differences, sun damage, smoking, radiodermatitis and vitamin A deficiency.

Traditionally, large pores are treated by targeting their underlying associated causes, such as increased sebum production and skin aging, and by unclogging pores through exfoliation and peeling.

Large pores can be made to appear smaller by decreasing or regulating sebum production and counteracting skin aging. Exfoliation can also help by removing clogs and reducing dilation. Retinoids and anti-aging solutions may be utilized to counteract skin aging and encourage firm and elastic skin. Scrubs, cleansers and face masks lightly exfoliate the skin, whereas chemical peels provide a deep exfoliation and can also decrease sebum production.

A topical retinoid is a vitamin A derivative, which helps reverse the skin changes that occur with aging and sun damage by increasing skin thickness and elasticity and by slowing collagen breakdown. Retinoids accelerate cellular turnover and force excess sebum out of pores. However, retinoids can sometimes cause side effects such as burning, scaling, peeling, redness and swelling that make them unpleasant to use.

A chemical peel is a solution which removes and regenerates the outer layers of skin, acting as a much deeper exfoliant than face scrubs and cleansers. This exfoliation forces sebum out of the pores, reducing the volume within them and therefore diminishes their appearance. Chemical peels have proven effective at treating large pores. However, those with rosacea, dark skin tones and sensitive skin may experience some irritation from chemical peel use.

Therefore, there remains a need for an effective treatment of large pores avoiding skin irritation and other unpleasant side effects.

The present invention provides, in a first aspect, a cosmetic active agent for the treatment of large pores.

The cosmetic active agent comprises:
(i) a mixture of mannose-6-phosphate and mannose, wherein the molar ratio of mannose-6-phosphate to mannose is from 3:1 to 0.3:1;
(ii) copper ions;
(iii) a first amino acid selected from the group consisting of lysine, arginine, histidine, and mixtures thereof;
(iv) a second amino acid selected from the group consisting of proline, aspartic acid, glutamic acid, and mixtures thereof; and
(v) an acid selected from the group consisting of lactic acid and 2-pyrrolidone-5-carboxylic acid.

In a second aspect, the present invention provides a cosmetic composition comprising the cosmetic active agent and a cosmetically acceptable excipient. In particular, the invention relates to a skin care composition.

In a third aspect, the present invention provides a method of reducing the number and/or size of pores in human skin, comprising the step of applying the cosmetic active agent or the cosmetic composition to the human skin. This is particularly useful for facial skin.

In a fourth aspect, the present invention provides a cosmetic method of reducing parakeratosis in human skin, comprising the step of applying the cosmetic active agent or the cosmetic composition to the human skin. This is particularly useful for facial skin.

In a fifth aspect, the present invention provides a cosmetic method of restoring the epidermal integrity of human skin, comprising the step of applying the cosmetic active agent or the cosmetic composition to the human skin. This is particularly useful for facial skin.

In a sixth aspect, the present invention provides a method of boosting collagen synthesis in human skin, comprising the step of applying the cosmetic active agent or the cosmetic composition to the human skin. This is particularly useful for facial skin.

Surprisingly, it has been found that the cosmetic active agent of the invention is highly effective against enlarged pores and significantly reduces the pore area. At the same time, it avoids the undesirable side effects known from the active of the prior art.

The cosmetic active agent of the invention comprises several known components, which interact in a synergistic manner and provide the hitherto unknown effect of pore reduction. This effect has previously not been associated with any of the contained components, nor was there any hint or suggestion in the prior art to this end.

The cosmetic active agent of the invention comprises a mixture of mannose-6-phosphate and mannose, wherein the molar ratio of mannose-6-phosphate to mannose is from 3:1 to 0.3:1.

A cosmetic active ingredient comprising such a mixture has been previously described in WO 2020/201185 in the context of anti-ageing. The contents of WO 2020/201185 are herewith incorporated by reference, in particular with regard to the synthesis of the ingredient.

The cosmetic active agent of the present invention may comprise D-mannose-6-phosphate, L-mannose-6-phosphate, or a mixture thereof. Preferably, it comprises D-mannose-6-phosphate.

Likewise, the cosmetic active agent of the present invention may comprise D-mannose, L-mannose, or a mixture thereof. Preferably, it comprises D-mannose.

Throughout this application, if not specified differently, the terms “mannose-6-phosphate” and “mannose” are meant to encompass both the D- and L-forms, as well as mixtures thereof.

In the cosmetic active agent of the present invention, mannose-6-phosphate may be present in any cosmetically acceptable form. For instance, depending on the pH, mannose-6-phosphate may be present in protonated form or in the form of a salt. Suitable counter ions include, but are not limited to, monovalent cations, such as e.g. sodium, potassium, or ammonium; divalent cations, such as e.g. copper, zinc, calcium, magnesium, or manganese; or trivalent cations, such as e.g. aluminum; or mixtures thereof. Mannose-6-phosphate may also be mixed with one or more cosmetically acceptable positively charged substance(s), and may form a salt with said cosmetically acceptable positively charged substance(s).

Throughout this application, if not specified differently, the term “mannose-6-phosphate” is meant to encompass not only the free form, but also the protonated form and any cosmetically acceptable salt of mannose-6-phosphate, as well as mixtures thereof.

The cosmetic active agent of the invention further comprises copper ions. Within the cosmetic active agent of the invention, these copper ions are mostly or exclusively present in the form of Cu²⁺ ions. However, during the preparation of the cosmetic active agent, copper ions may be added in the form of Cu²⁺ ions and/or Cu⁺ ions, with (part of) the latter subsequently being oxidized to form Cu²⁺ ions.

The cosmetic active agent of the invention further comprises a first and a second amino acid.

Without being bound by theory, it is believed that the first and/or second amino acids are associated with the copper ions, possibly by means of ionic bonds and/or forming a complex-like structure.

Throughout this application, the term “amino acid” is meant to encompass not only the free form of the amino acid, but also a close derivative thereof, such as a salt, an ester, an amide, an N-acetylate or a hydroxamate.

The first amino acid used in the cosmetic active agent of the invention has a basic side chain. In an embodiment, the first amino acid is selected from the group consisting of lysine, arginine, histidine, and mixtures thereof.

The second amino acid used in the cosmetic active agent of the invention has an acidic side chain. Alternatively, the second amino acid may be proline. In an embodiment, the second amino acid is selected from the group consisting of proline, aspartic acid, glutamic acid, and mixtures thereof.

The cosmetic active agent of the invention further comprises an acid. Said acid may serve several purposes, e.g. adjustment of the pH and/or for moisturizing properties. In an embodiment, the acid is selected from the group consisting of lactic acid, malic acid, succinic acid, fumaric acid, maleic acid, pyruvic acid, citric acid, gluconic acid, lactobionic acid, sorbic acid, tartaric acid, oxalic acid, 2-pyrrolidone-5-carboxylic acid, and mixtures thereof. Preferably, the acid is selected from the group consisting of lactic acid and 2-pyrrolidone-5-carboxylic acid.

The cosmetic active agent of the invention is able to significantly reduce the number and/or size of pores in human skin.

The cosmetic active agent of the invention is also able to reduce parakeratosis, to restore epidermal integrity and to boost collagen synthesis in human skin.

These effects are particularly advantageous for visible areas of the skin, especially in the face.

Mannose-6-phosphate may be prepared from mannose by an enzymatic phosphorylation. Suitable phosphorylation conditions are described, for instance, in WO 2008/142155; and example 1 of WO 2020/201185 describes a possible synthesis of mannose-6-phosphate in detail. The contents of these two disclosures in this respect are herewith incorporated by reference.

The enzymatic phosphorylation typically provides a mixture of mannose-6-phosphate and mannose. Depending on the reaction time and other conditions, the conversion and thus the ratio of mannose-6-phosphate to mannose may vary. Thus, preferably, the reaction time and conditions are chosen such that the desired mannose-6-phosphate to mannose ratio is obtained directly. Alternatively, it is also possible to adjust the ratio by adding or removing one or both of the products.

In an embodiment, the molar ratio of mannose-6-phosphate to mannose is from 2:1 to 1:1, more preferably from 1.9:1 to 1.1:1, in particular about 1.5:1. It has been found that these ratios are particularly advantageous.

In an embodiment, the cosmetic active agent of the invention comprises mannose-6-phosphate in a concentration of 30 to 220 mM, more preferably in a concentration of 60 to 170 mM, and most preferably in a concentration of about 120 mM.

In particular, the cosmetic active agent of the invention may comprise from 0.5 to 6.0 wt % of mannose-6-phosphate sodium salt, more preferably from 2.0 to 4.0 wt % of mannose-6-phosphate sodium salt, and most preferably about 3.0 wt % of mannose-6-phosphate sodium salt. Alternatively, the cosmetic active agent of the present invention may comprise mannose-6-phosphate in any other form described above, in corresponding amounts.

In an embodiment, the cosmetic active agent of the invention comprises from 0.5 to 5.0 wt % of mannose, more preferably from 0.8 to 3.0 wt % of mannose, and most preferably about 1.5 wt % of mannose.

The copper ions present in the cosmetic active agent of the invention may be provided in any suitable form upon preparation of the cosmetic active agent, for example in the form of a Cu²⁺ and/or Cut salt. Preferably, they are added in the form of a Cu²⁺ salt. The counter ion(s) used in said copper salt may or may not be directly associated with the copper ions in the cosmetic active agent of the invention once it has been prepared.

Therefore, in an embodiment, the copper ions are provided as a copper salt selected from the group consisting of copper sulphate, copper phosphate, copper carbonate, copper chloride, copper acetate, copper malate, copper succinate, copper fumarate, copper maleate, copper pyruvate, copper citrate, copper gluconate, copper glucuronate, copper lactobionate, copper sorbate, copper tartrate, copper oxalate, copper lactate, copper pyroglutamate, copper prolinate, copper aspartate, copper glutamate, and mixtures thereof, more preferably as copper sulphate. It has been found that these copper salts are particularly suitable for use in cosmetic compositions and allow for forming a stable product.

In an embodiment, the cosmetic active agent of the invention comprises copper ions in a concentration of about 10 mM to about 40 mM, more preferably of about 12 mM to about 30 mM, and most preferably of about 12.5 mM to about 25 mM. For example, the cosmetic active agent may comprise copper ions in a concentration of 12.5 mM to 25.1 mM.

In an embodiment, the first amino acid comprises or consists of lysine.

In an embodiment the second amino acid comprises or consists of proline.

Preferably, first amino acid comprises or consists of lysine and the second amino acid comprises or consists of proline.

The cosmetic active agent of the invention may comprise the first and the second amino acid in any suitable ratio.

In an embodiment, the cosmetic active agent of the invention comprises the first amino acid and the second amino acid in a molar ratio of about 3:5 to about 5:2, more preferably of about 9:10to about 10:6, and most preferably of about 93:100 to about 100:63. For example, the cosmetic active agent of the invention may comprise lysine and proline in a molar ratio of about 0.071:0.119 to about 0.120:0.049, more preferably of about 0.0886:0.0955 to about 0.0958:0.0608.

In an embodiment, the cosmetic active agent of the invention comprises the first amino acid in a concentration of about 50 mM to about 120 mM, more preferably of about 60 mM to about 100 mM, and most preferably of about 61 mM to about 96 mM, for example about 78 mM.

In an embodiment, the cosmetic active agent of the invention comprises the second amino acid in a concentration of about 50 mM to about 120 mM, more preferably of about 60 mM to about 100 mM, and most preferably of about 61 mM to about 96 mM, for example about 78 mM.

The acid contained in the cosmetic active agent of the invention may be used for adjusting the pH of the cosmetic active agent to a cosmetically acceptable level.

Therefore, in an embodiment, the cosmetic active agent of the invention comprises the acid in an amount for the cosmetic active agent to have a pH of about 3.8 to about 6.0, more preferably of about 4.5 to about 5.3. The person skilled in the art knows how to determine the required amounts.

The cosmetic active agent of the invention may comprise further ingredients that support the desired effects or provide other benefits.

Thus, the cosmetic active agent of the invention may optionally further contain other cosmetically active ingredients. Any cosmetically active ingredients commonly used in the preparation of cosmetic preparations for use on the human skin may be employed in the present invention.

The cosmetic active agent of the invention may optionally further contain solvents, excipients, and/or other adjuvants. Any solvents, excipients, and/or other adjuvants commonly used in the preparation of cosmetic preparations for use on the human skin may be employed in the present invention.

In particular, the cosmetic active agent of the present invention may further comprise 1,2-propanediol, 1,3-propanediol, 1,3-butanediol, 2,3-butanediol, 1,4-butanediol, and/or glycerol. They may serve as a preservative, for instance.

Alternatively or in addition, the cosmetic active agent of the present invention may further comprise sodium phosphate and/or sodium hydroxide. Sodium phosphate may be used as a buffer, for instance.

In an embodiment, the cosmetic active agent of the invention further comprises another amino acid, wherein the amino acid is selected from the group consisting of glutamine, asparagine, glycine, hydroxyproline, serine, methionine, threonine, and mixtures thereof.

In a preferred embodiment, the cosmetic active agent of the invention comprises:

| | |
|---|---|
| 2.5-3.5 wt % | mannose-6-phosphate sodium salt |
| 0.7-1.7 wt % | mannose |
| 0.7-1.1 wt % | lysine |
| 1.0-1.4 wt % | proline |
| 0.2-0.4 wt % | copper sulphate |
| 0.8-1.5 wt % | lactic acid |

The cosmetic active agent of the present invention is advantageously used in cosmetic compositions, in particular in skin care compositions.

Therefore, in a further aspect, the present invention provides a cosmetic composition, and in particular a skin care composition, comprising the above described cosmetic active agent and a cosmetically acceptable excipient. Preferably, cosmetic composition comprises the cosmetic active agent in the preferred embodiments outlined herein.

Typically, the cosmetic active agent of the invention will be used in a concentration of about 0.1 to 5.0 wt % in the cosmetic composition, more preferably in a concentration of about 0.5 to 3.0 wt %, for instance in a concentration of about 1 wt %.

The cosmetic composition of the present invention comprises a cosmetically acceptable excipient.

Cosmetic compositions, and in particular skin care compositions, of the present invention may contain one or more cosmetically acceptable excipients. Any excipients commonly used in the preparation of cosmetic preparations for use on the human skin may be employed in the present invention. Suitable excipients include, but are not limited to ingredients that can influence organoleptic properties, penetration of the skin, and the bioavailability of the cosmetic active agent of the present invention. More specifically, they include liquids, such as water, oils or surfactants, including those of petroleum, animal, plant or synthetic origin, such as and not restricted to, peanut oil, soybean oil, mineral oil, sesame oil, castor oil, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, digitonin, and the like.

The formulation for topical application to the skin may take any physical form. For instance, the cosmetic composition, and in particular the skin care composition, may be in the form of a liposome composition, mixed liposomes, oleosomes, niosomes, ethosomes, milliparticles, microparticles, nanoparticles and solid-lipid nanoparticles, vesicles, micelles, mixed micelles of surfactants, surfactant-phospholipid mixed micelles, millispheres, microspheres and nanospheres, lipospheres, millicapsules, microcapsules and nanocapsules, as well as microemulsions and nanoemulsions, which can be added to achieve a greater penetration of the cosmetic active agent of the present invention.

The cosmetic composition, and in particular the skin care composition, may be produced in any solid, liquid, or semi-solid form useful for application to the skin topically or by transdermal application. Thus, these preparations of topical or transdermal application include, but are not restricted to, creams, multiple emulsions, such as and not restricted to, oil and/or silicone in water emulsions, water-in-oil and/or silicone emulsions, water/oil/water or water/silicone/water type emulsions, and oil/water/oil or silicone/water/silicone type emulsions, micro-emulsions, emulsions and/or solutions, liquid crystals, anhydrous compositions, aqueous dispersions, oils, milks, balsams, foams, aqueous or oily lotions, aqueous or oily gels, cream, hydro-alcoholic solutions, hydro-glycolic solutions, hydrogels, liniments, sera, soaps, face masks, serums, polysaccharide films, ointments, mousses, pomades, pastes, powders, bars, pencils and sprays or aerosols (sprays), including leave-on and rinse-off formulations.

In order to achieve the beneficial effects described herein, the cosmetic active agent or cosmetic composition of the invention is advantageously applied to the skin, in particular to facial skin.

Throughout this application, the term “skin” refers in particular to human skin.

In a further aspect, the present invention provides a method of reducing the number and/or size of pores in human skin.

One of the factors responsible for pore formation is the alteration of epidermal integrity linked to a stimulation of parakeratosis, leading to an impaired epidermal renewal and strong accumulation of dead cells at the skin surface obstructing the pore.

It has been found that by applying the cosmetic active agent of the invention or the cosmetic composition of the present invention to the skin, it is possible to reduce the pore area in skin, in particular in facial skin.

It has further been found that by applying the cosmetic active agent of the invention or the cosmetic composition of the present invention to the skin, it is possible to reduce parakeratosis.

It has still further been found that by applying the cosmetic active agent of the invention or the cosmetic composition of the present invention to the skin, it is possible to restore the epidermal integrity.

It has still further been found that by applying the cosmetic active agent of the invention or the cosmetic composition of the present invention to the skin, it is possible to boost collagen synthesis.

These effects have been confirmed by in vitro, ex vivo and clinical studies as described in the examples below.

The present invention also relates to the use of the cosmetic active agent or the cosmetic composition of the present invention for any of the above purposes.

The present invention is further illustrated by means of the following non-limiting examples:

### EXAMPLE 1

### Preparation of Cosmetic Active Agent

The procedure for preparing 1.00 kg of a cosmetic active agent according to the invention is as follows:
1) In an agitated and thermostated reactor, 125 g of demineralized water was introduced. The water was agitated mildly and the temperature was maintained at a value ranging from 18° C. to 28° C., preferably 23° C.
2) 9.4 g of proline (C₅H₉NO₂; 115.13 g/mol; CAS: 147-85-3) was introduced into the reactor and dispersed completely under mild agitation.
3) 2.84 g of copper sulfate (CuSO₄; 159.61 g/mol; CAS: 7758-98-7) was introduced into the reactor and dispersed completely under mild agitation.
4) 11.9 g of lysine (C₆H₁₄N₂O₂; 146.19 g/mol; CAS: 56-87-1) was introduced into the reactor and dispersed completely under mild agitation; a deep blue solution was obtained. The pH of the solution was 9.45 (a value ranging from 9.0 to 10.0, preferably from 9.3 to 9.6,was considered as acceptable).
5) 27.6 g of mannose-6-phosphate (C₆H₁₃O₉P; 260.14 g/mol; CAS: 3672-15-9) and 14.4 g of mannose (C₆H₁₂O₆; 180.16 g/mol; CAS 3458-28-4) were introduced into the reactor. These substances were added together in the form of an aqueous solution (a suitable mixture comprises mannose-6-phosphate at a concentration ranging from 0.065 kg/kg to 0.098 kg/kg and mannose at a concentration of from 0.031 kg/kg to 0.047 kg/kg). In the present instance, an aqueous solution containing mannose-6-phosphate at a concentration of 0.0813 kg/kg and mannose at a concentration of 0.0424 kg/kg was used and, thus, the amount of solution added was 339.5 g.
6) 14.9 g of lactic acid (C₃H₆O₃; 90.08 g/mol; CAS: 79-33-4) was introduced into the reactor and dispersed completely under mild agitation. The pH of the mixture was measured and a value of 3.25 was obtained (a value ranging from 2.9 to 3.6, preferably from 3.1 to 3.4, was considered as acceptable).
7) The pH was adjusted to a value of 4.65 with 2 N sodium hydroxide (sodium hydroxide: CAS 1310-73-2) (a value ranging from 4.2 to 5.1, preferably from 4.5 to 4.8, was considered as acceptable).
8) The weight of the mixture was adjusted to 750 g by addition of demineralized water.
9) The weight of the mixture was adjusted to 1.00 kg by addition of 1,3-propanediol (C₃H₈O₂; 76.09 g/mol; CAS 504-63-2).
10) The mixture was stored in stoppered sterile bottle at cold temperature (4° C. to 10° C.).

The cosmetic active agent obtained by this process had the following composition and a pH of about 5.06:

| Ingredient | Concentration (wt %) |
|---|---|
| Proline | 0.94 |
| Copper sulphate | 0.28 |
| Lysine | 1.19 |
| Mannose | 1.44 |
| Sodium mannose-6-phosphate | 2.99 |
| Lactic acid | 1.18 |
| 1,3-propanediol | 25 |
| Water | Qs 100 |

### EXAMPLE 2

### Skin Care Composition Comprising the Cosmetic Active Agent

For the clinical studies described in example 9 below, the following compositions were prepared:

| | Concentration (wt %) | |
|---|---|---|
| | Active | Placebo |
| Ingredient (INCI Name) | Cream | Cream |
| AQUA/WATER | 88.1 | 89.1 |
| CETHYL ALCOHOL, | 5.0 | 5.0 |
| GLYCERYL STEARATE, PEG-75 | | |
| STEARATE, CETEH-20, STEARETH-20 | | |
| ISODECYL NEOPENTANOATE | 4.5 | 4.5 |
| Cosmetic Active Agent of Example 1 | 1.0 | — |
| PHENOXYETHANOL | 1 | 1 |
| DIMETHICONE | 0.3 | 0.3 |
| FRAGRANCE | 0.1 | 0.1 |

For the clinical studies described in example 10 below, the following compositions were prepared:

| | Concentration (wt %) | |
|---|---|---|
| | Active | Placebo |
| Ingredient (INCI Name) | Cream | Cream |
| AQUA/WATER | 88.3 | 89.3 |
| CETHYL ALCOHOL, GLYCERYL | 5.0 | 5.0 |
| STEARATE, PEG-75 STEARATE, | | |
| CETEH-20, STEARETH-20 | | |
| ISODECYL NEOPENTANOATE | 4.5 | 4.5 |
| Cosmetic Active Agent of Example 1 | 1.0 | — |
| PHENOXYETHANOL | 0.5 | 0.5 |
| 1.2-HEXANEDIOL, CAPRYLYL | 0.3 | 0.3 |
| GLYCOL | | |
| DIMETHICONE | 0.3 | 0.3 |
| FRAGRANCE | 0.1 | 0.1 |

### EXAMPLE 3

### Statistical Analysis

For the in vitro and ex vivo studies described below, all results are presented as mean + standard error of mean (SEM) of three independent triplicates. For all studies, a Shapiro Wilk test was used to verify whether the raw data followed the Gaussian Law. In case of normally-distributed data, the mean values were compared using either an unpaired or paired t test (≤2 groups) or a one-way ANOVA followed by post-hoc test (≥2 groups). In case of non-normally-distributed data, a Wilcoxon (paired) and Kruskal-Wallis test followed by a Mann-Whitney U (unpaired) test were used for paired data and unpaired data, respectively.

Results were considered significant as follows: #p<0.1, *p<0.05, **p<0.01 and ***p<0.001.

### EXAMPLE 4

### Transcriptomic Study on Keratinocytes

### Materials and Methods

Normal Human Epidermal Keratinocytes (NHEKs) were seeded at 300′000 cells per well in a 6-wells plate. After 48 h of culture, NHEKs were rinsed two times with phosphate-buffered saline (PBS) and allowed to rest in a factors-free medium overnight before stimulation.

Cells were stimulated with the Cosmetic Active Agent of Example 1 at 0.5%. After 24 h of stimulation, total RNA was extracted by the “TRIzol method” (Rio DC, Ares M Jr, Hannon G J, Nilsen T W. Purification of RNA using TRIzol (TRI reagent). Cold Spring Harb Protoc. 2010 Jun;2010(6):pdb.prot5439. doi: 10.1101/pdb.prot5439. PMID: 20516177.). RNA quality was controlled and a reverse transcription was performed to obtain cDNA.

RT-qPCR was made on specific plates designed to study transcriptomic expression of different genes involved in epidermis biology for NHEKs with 10 ng of cDNA per well.

The results of gene expression obtained with keratinocytes were normalized according to EIF2B1 (Eukaryotic Translation Initiation Factor 2B Subunit Alpha) and ABL1 (ABL Proto-20) Oncogene 1, Non-Receptor Tyrosine Kinase) housekeeping gene.

### RESULTS

The transcriptomic analysis was performed by RT-qPCR on plates with targeted genes involved in desquamation, differentiation and stem cells markers. The results are expressed in comparison to an untreated condition used a negative control and normalized with the average of the most stable housekeeping genes (EIF2B1 and ABL1).

A significant regulation of 7 genes by the treatment the cosmetic active agent of the invention was observed. The results are shown in the following table:

| Pathway | Genes | Fold change | SEM | p value | |
|---|---|---|---|---|---|
| Stem cells niche | CTNNB1 | 1.335 | 0.011 | <0.0001 | *** |
| | ITGA6 | 1.181 | 0.011 | <0.0001 | *** |
| | ITGB1 | 1.213 | 0.028 | 0.005 | ** |
| Differentiation | LOR | −1.631 | 0.024 | <0.0001 | *** |
| | SPRR3 | −1.185 | 0.023 | 0.006 | ** |
| | CDSN | −1.347 | 0.088 | 0.091 | # |

Specifically, the cosmetic active agent significantly upregulated the KLK7 gene, which is involved in the skin desquamation process. It also decreased the expression of genes involved in differentiation, such as LOR, SPRR3 and CDSN. Finally, the cosmetic active agent upregulated CTNNB1, ITGA6 and ITGB1, which are involved in stem cells niche.

These various effects suggest that the cosmetic active agent of the invention could bring a clear benefit in a context of parakeratosis where there is an accumulation of stratum corneum due to a poor skin renewal and desquamation processes.

### EXAMPLE 5

### Evaluation of Parakeratosis

### Skin Explants Culture and Treatment

Reconstructed Human Epidermis (RHE) was cultivated in an air-liquid interface and was pre-treated for 24 h with the Cosmetic Active Agent of Example 1 at 1%. RHE was stressed for 48 h with oleic acid at 2.5% to induce an epidermis alteration mimicking parakeratosis, in the presence or absence of 1% of the Cosmetic Active Agent of Example 1. RHE was fixed in formaldehyde solution. Fixed samples were dehydrated in successive ethanol baths of increasing concentrations before being embedded in paraffin.

### Parakeratosis Evaluation

Transversal sections were performed using a microtome (5 μm thickness, 2 sections per slide, 1slide per RHE) and kept at room temperature until analysis. Tissue sections were deparaffinized and stained according to the standard protocol of HE staining: Briefly, sections were stained with hematoxylin, rinsed and stained with eosin. Sections were then rinsed and mounted in aqueous medium.

To evaluate parakeratosis, the number of nuclei in the stratum corneum was counted on a defined length of RHE section.

### Involucrin Immunostaining

Tissue sections were deparaffinized and incubated at 95° C. in an unmasking solution to optimize antigen-antibody interaction. Slides were cooled down to room temperature in the same solution. After saturation with TBS-Tween-2% BSA (TRIS BASE solution containing Tween and 2% of bovin serum albumin), tissue sections were incubated overnight with the primary antibody solution directed against the marker of interest (Involucrin).

After washing, binding sites recognized by the primary antibody were revealed by a secondary fluorescent antibody (GAR-568). Labelled tissue sections were mounted with Prolong® containing DAPI (4′,6-diamidino-2-phenylindole) to color nuclei. Labelled tissue sections were observed using a ZEISS 710 confocal microscope. Images were captured and processed with ZEN software (objective lens x20).

### Results Parakeratosis Evaluation

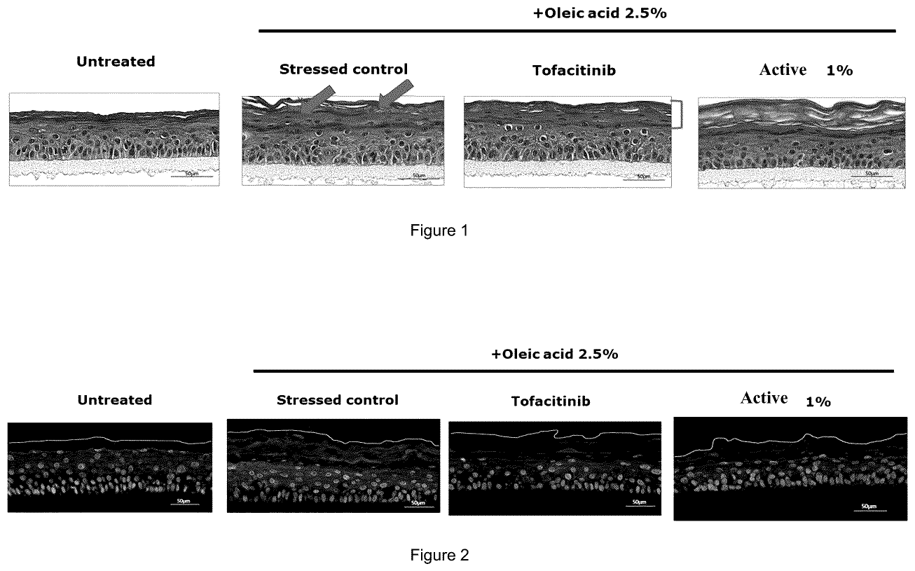

FIG. 1 shows the HE staining of RHE showing the nuclei in the stratum corneum. The number of nuclei counted is shown in the following table:

### Reduction of Parakeratosis (Number of Nuclei in Stratum Corneum)

| | | | Parakeratosis + |
|---|---|---|---|
| | Untreated | Parakeratosis | Neoporyl 1% |
| Average | 0 | 16.67 | 7.833 |
| SEM | 0 | 2.565 | 1.99 |
| p value/ | | 0.0119* | |
| Untreated | | | |
| p value/ | | | 0.0206* |
| Parakeratosis | | | |

As can be seen from FIGS. 1 and 2, oleic acid treatment significantly increased the number of nuclei in the stratum corneum, a marker of parakeratosis. Placebo control did not improve this phenomenon, while the cosmetic active agent of the invention at 1% significantly reduced the parakeratosis by −53%, as observed by decreasing nuclei number.

### Results Involucrin Immunostaining

FIG. 2 shows the involucrin immunostaining in RHE mimicking parakeratosis, and the following table shows the quantification of staining intensity.

### Limitation of Hyperdifferentiation (Involucrin Expression in % of Parakeratosis Condition)

| | | | Parakeratosis + |
|---|---|---|---|
| | Untreated | Parakeratosis | Neoporyl 1% |
| Average | 27.33 | 100 | 62.17 |
| SEM | 7.688 | 8.836 | 12.7 |
| p value/ | | 0.0119* | |
| Untreated | | | |
| p value/ | | | 0.0152* |
| Parakeratosis | | | |

As can be seen from FIGS. 3 and 4, involucrin immunostaining confirmed the restoration of epidermal integrity as shown by the decrease of involucrin expression by −38% with the cosmetic active agent of the invention at 1%.

### EXAMPLE 6

### Dermis Fragility: Transcriptomic Study on Fibroblasts

### Materials and Methods

Normal Human Dermal Fibroblasts NHDFs were seeded at 300'000 cells per well in a 6-wells plate. After 48 h of culture, a premature ageing was induced with H₂O₂ treatment at 200 μM for 2 h versus basal condition. NHDFs were then rinsed two times with PBS and allowed to rest in an FCS-free medium overnight before stimulation.

Cells were stimulated with the Cosmetic Active Agent of Example 1 at 0.5% in basal and in premature ageing (H₂O₂) condition. After 24 h of stimulation, total RNA was extracted by the “Extract-all method”. RNA quality was controlled and a reverse transcription was performed to obtain cDNA.

RT-qPCR was made on specific plates designed to study transcriptomic expression of different genes involved in dermis biology for NHDFs with 10 ng of cDNA per well. The results of gene expression obtained with fibroblasts were normalized according to PES1 (Pescadillo Ribosomal Biogenesis Factor 1) and GAPDH (Glyceraldehyde-3-Phosphate Dehydrogenase) housekeeping gene in basal condition and according to HMBS (Hydroxymethylbilane Synthase) and GAPDH in premature aged condition.

### RESULTS

The results are shown in the following table:

| Pathway | Genes | Fold change | SEM | p value | |
|---|---|---|---|---|---|
| Dermis structure | COL3A1 | 1.185 | 0.027 | 0.008 | ** |
| | MMP1 | −2.361 | 0.023 | <0.0001 | *** |
| Matrix degradation | MMP13 | −1.997 | 0.161 | 0.076 | # |
| | MMP8 | −1.528 | 0.055 | 0.01 | * |
| Skin homeostasis | ADAM2 | 1.415 | 0.075 | 0.015 | * |

In the basal condition, it was found that the cosmetic active agent of the invention significantly upregulated COL3A1 and LOXL4 genes involved in dermis structure through their role in collagen and elastin fibers organization. It also decreased the expression of genes involved in matrix degradation, such as MMP1, MMP13 and MMP3.

After a premature ageing, the cosmetic active agent of the invention also upregulated COL3A1,showing an improvement of the dermis structure even in this condition, and it also protected the matrix from degradation by decreasing the expression of MMP1, MMP13 and MMP8 genes. Additionally, the cosmetic active agent of the invention upregulated the ADAM2 gene, showing a benefit regarding skin homeostasis.

These results showed that the cosmetic active agent of the invention could protect dermis against degradation and could be a good active to protect skin against dermis fragility, which is a factor involved in pores formation.

### EXAMPLE 7

### Evaluation of Collagen Stimulation: Stimulation ex vivo Following Dermis Fragility

### Skin Explants Culture and Treatment

Human fresh skin explants were obtained from a 31 years-old donor.

Skin explants were topically pre-treated with the Cosmetic Active Agent of Example 1 at 1% (v/v) for 3 days. The untreated condition was treated with the vehicle (distilled water).

Dermis weakening was induced by an overnight enzymatic degradation, and skin explants were post-treated for 3 days. The medium was renewed every other day. After the end of culture, the RNA from the skin was extracted and reverse-transcribed into cDNA. The expression of COL1A1, COL5A1, COL7A1, COL17A1 mRNA was measured by semi-quantitative PCR on the Applied Biosystems 7300 Real Time PCR System. The expression level of the mRNAs was calculated and normalized with reference genes (GAPDH).

### RESULTS

As can be seen from the table below, it was found that the enzymatic collagen degradation led to a significant reduction of collagens I, V, VII and XVII, and they were significantly restored by the topical application of the cosmetic active agent of the invention at 1% by +31%, 31%, 45% and 35% for the collagens I, V, VII and XVII, respectively.

| | | Enzymatic | Enzymatic degradation + |
|---|---|---|---|
| | | degradation | Neoporyl 1% |
| Collagen I | Average | 40.4 | 71.8 |
| | SEM | 8.8 | 11 |
| | p value/Enzymatic | | 0.02 * |
| | deg. | | |
| Collagen V | Average | 16.5 | 47.6 |
| | SEM | 1.4 | 8.5 |
| | p value/Enzymatic | | 0.001 ** |
| | deg. | | |
| Collagen VII | Average | 27.9 | 72.7 |
| | SEM | 5.7 | 8.9 |
| | p value/Enzymatic | | 0.01 * |
| | deg. | | |
| Collagen | Average | 16.6 | 51.1 |
| XVII | SEM | 2.9 | 5.9 |
| | p value/Enzymatic | | 0.0004 *** |
| | deg. | | |

### EXAMPLE 8

### Evaluation of Collagen Stimulation: In vitro Organization of Collagen Fibers

### Materials and Methods

The cell culture was done on primary cells isolated from biopsies. Normal Human Dermal Fibroblasts (NHDFs) were seeded in a 96 wells black plate at 10′000 cells per well in triplicate. The cells were incubated for 48 h in DMEM medium supplemented with 10% fetal calf serum and 1% antibiotics at 37° C. with 5% CO₂. At the end of the incubation, the NHDFs were stressed with H₂O₂ at 200 μM for 2 h at 37° C. with 5% CO₂ and were then rinsed twice with PBS and incubated for 72 h in basal medium (DMEM medium without FCS) supplemented with 1% antibiotics containing the Cosmetic Active Agent of Example 1 at 1% versus TGF-β at 10 ng/ml+Vitamin C at 20 μg/ml for the positive control of pro-collagen I synthesis.

At the end of the culture, cells were fixed for 5 min with a paraformaldehyde (PAF) solution at 2% and were then permeabilized for 15 min with PAF solution at 2% supplemented with 0.5% Triton X100. Nonspecific sites were saturated with 3% bovine serum albumin (BSA), and primary antibodies targeting Collagen I and Decorin were incubated overnight at 4° C. On the next day, cells were rinsed and incubated with secondary antibodies coupled to Alexa Fluor 488 for Collagen I and to Alexa fluor 568 for Decorin. Fluorescent pictures and detection were automatically taken with PICO tool (Molecular Devices).

### RESULTS

The results are shown in the following table:

| | | Oxidative | Oxidative stress + |
|---|---|---|---|
| | | stress | Neoporyl 1% |
| Mature | Average | 100 | 121.7 |
| collagen | SEM | 3.51 | |
| | p value/Ox. | | 6.96 |
| | Stress | | 0.05 * (p < 0.05) |
| Decorin | Average | 100 | 152.7 |
| | SEM | 11.57 | 19.38 |
| | p value/Ox. | | 0.05 * (p < 0.05) |
| | Stress | | |

The cosmetic active agent of the invention at 1% significantly stimulated the collagen I synthesis in aged-induced condition as previously shown with the dosage of pro-collagen I in the fibroblasts culture medium.

The expression of decorin was also significantly improved in aged-induced condition, demonstrating an impact on collagen organization in the presence of the cosmetic active agent of the invention at 1%.

### EXAMPLE 9

### Clinical Evaluation: Impact on Enlarged Pores in Caucasian Volunteers

### Panel Description

A clinical study was carried out in double blind and placebo controlled condition on one group of 33 volunteers aged between 27 to 66 years (mean age 44), divided in two subgroups of 20- to 30-years-old (17 volunteers) and over 50-years-old (16 volunteers). The 33 volunteers were divided into two groups with homogenous distribution according to their age in order to do an evaluation of global cohort but also by range of age.

Volunteers were recruited according to the following inclusion criteria: having visible pores and a lack of skin luminosity, dull complexion on the face. All the subjects participating in the study gave their informed consent signed at the beginning of the study. The study followed and was in compliance with the tenets of the Declaration of Helsinki.

Volunteers applied twice a day an Active Cream containing 1% of the Cosmetic Active Agent of Example 1 on one hemi face and a Placebo Cream on the other hemi face for 56 days. The compositions of these creams are given in Example 2 above.

The reduction of enlarged pores was analyzed using VisioFace® based on total pore area quantification.

### Enlarged Pore Reduction Measured by VisioFace®

This technique consists of obtaining high resolution photographs of the ¾ face, in completely reproducible lighting conditions, in cross polarized light and in diffuse light.

The acquisitions are carried out with a high-resolution camera. The lens used is a Nikkor 60 mm equipped with the filter. Lighting is provided by two flash lights. The flash heads are fitted with filter slots to hold polarizing gel (HN32 Sarelec, France).

To obtain cross polarized light, the filter on the lens of the camera is positioned at 90° compared to the polarization of the filters of the flashes. The polarized light, emitted by the flashes and reflected by the skin of the face at the moment the photo is taken, is “cut” by the camera filter, i.e. these reflections do not appear on the photograph.

To obtain diffuse light photos, the filter on the lens of the camera is turned to 45° compared to the position for cross polarized light.

The analysis is carried out by a specific software developed by Spincontrol.

After the correction of the gradient and after the binarisation of the pictures, the backgrounds are deleted using a threshold.

The analysis is carried out on a determined area that is identical at each time of the study.

Total area of pores (mm²) extracted from the pictures is then calculated to measure the reduction of pore after applications of products and over time.

### RESULTS

The results for the global cohort are shown in table 1, those for the 20-30 year old volunteers in table 2, and those for the >50 year old volunteers in table 3 below.

In the global cohort, it was found that the presence of the cosmetic active agent of the invention at 1% significantly reduced the total pore area by −11.9%, −19% and −23.3% after 15, 28 and 56 days, respectively. Interestingly, the placebo formula did not have any effect on the surface of pore. It was further shown that the active cream is significantly better than the placebo, as observed by the significant reduction of total pore area in comparison with the placebo after 15, 28 and 56 days, respectively.

For the younger sub-group, it was found that the presence of the cosmetic active agent of the invention at 1% significantly reduced the total pore area by −10.3%, −19.5% and −24.3% after 15, 28 and 56 days, respectively. Interestingly, the placebo formula did not have any effect on the surface of pore after 15 and 56 days. It was further shown that the active cream is significantly better than the placebo, as observed by the significant reduction of total pore area in comparison with the placebo after 15, 28 and 56 days, respectively.

For the older sub-group, it was found that the presence of the cosmetic active agent of the invention at 1% significantly reduced the total pore area by −13.6%, −18.7% and −22.3% after 15, 28 and 56 days respectively. Interestingly, the placebo formula did not have any effect on the surface of pore. It was further shown that the active cream is significantly better than the placebo, as observed by the significant reduction of total pore area in comparison with the placebo after 15, 28 and 56 days, respectively.

Overall, these results demonstrated that the cosmetic active agent of the invention is significantly effective against enlarged face pores whatever the age of the volunteer. Indeed, a significant reduction of the total pore area was proven for both the younger and the older sub-group, demonstrating that the active is effective against the different sources of enlarged pore formation, including ageing.

### EXAMPLE 10

### Clinical Evaluation: Impact on Enlarged Pores in Asian Volunteers

### Panel Description

A clinical study was carried out in double blind and placebo controlled on 20 Asian men aged over 35 (mean age 42±10) divided into two groups, with 10 volunteers applying the Active Cream and the other 10 volunteers applying the Placebo Cream of Example 2.

Volunteers were recruited according to the following inclusion criteria: having visible enlarged pores on the face. All the subjects participating in the study gave their informed consent signed at the beginning of the study. The study followed and was in compliance with the tenets of the Declaration of Helsinki.

Volunteers applied twice a day either an Active Cream containing 1% of the Cosmetic Active Agent of Example 1 or a Placebo Cream on their entire face for 7 days.

The reduction of enlarged pores was analyzed by ColorFace® based on total pore area quantification.

### Enlarged Pore Reduction Measured by ColorFace®

Digital pictures of the face were done at different times with ColorFace® (Newtone). ColorFace® is a 2D acquisition system able to perform multimodal and standardized pictures of the face. This device is equipped to a captor of 24 Mpixel. There are various acquisition modes: Ultraviolet (UV) picture, without filter picture, cross polarized picture, 45° standard picture and 60° standard picture.

ColorFace® was used to analyze the pore surface at DO and after 7 days of treatment.

### RESULTS

The results are shown in table 4 below.

It was found that the total pore area was significantly reduced by −30.2% after only 7 days of application, while the placebo formula showed lower performance. Indeed, it was shown that the active performance on pore size reduction is significantly better than that of the placebo after 7days of application on Asian men.

Thus, the cosmetic active agent of the invention is effective against enlarged face pores on men of Asian ethnicity.

TABLE 1
(Caucasian, global cohort):

| | Active | | | Placebo | | | Unpaired |
|---|---|---|---|---|---|---|---|
| | Mean +/− SD | Average | | Mean +/− SD | Average | | t test |
| | (arbitrary | variation | paired t test | (arbitrary | variation | Paired t test | versus |
| | unit) | (%) vs D 0 | versus D 0 | unit) | (%) vs D 0 | versus D 0 | placebo |
| D 0 | 9204 ± 3248 | | | 8135 ± 3445 | | | |
| D 15 | 8252 ± 3286 | −11.9% | P < 0.001 *** | 8357 ± 3596 | +3.7% | ns | *** p < 0.001 |
| D 28 | 7446 ± 3264 | −19% | P < 0.001 *** | 8085 ± 3592 | −0.6% | ns | *** p < 0.001 |
| D 56 | 7055 ± 3139 | −23.3% | P < 0.001 *** | 8137 ± 3603 | +0.7% | ns | *** p < 0.001 |

TABLE 2
(Caucasian, 20-30 years old):

| | Active | | | Placebo | | | Unpaired |
|---|---|---|---|---|---|---|---|
| | Mean +/− SD | Average | | Mean +/− SD | Average | | t test |
| | (arbitrary | variation | paired t test | (arbitrary | variation | Paired t test | versus |
| | unit) | (%) vs D 0 | versus D 0 | unit) | (%) vs D 0 | versus D 0 | placebo |
| D 0 | 9537 ± 3125 | | | 8283 ± 2935 | | | |
| D 15 | 8901 ± 2983 | −10.3% | P < 0.01 ** | 8435 ± 3393 | +3.7% | ns | * p < 0.05 |
| D 28 | 7682 ± 3220 | −19.5% | P < 0.001 *** | 7491 ± 3051 | −9.6% | ** P < 0.01 | * p < 0.05 |
| D 56 | 7223 ± 2807 | −24.3% | P < 0.001 *** | 7809 ± 2361 | −4.5% | ns | *** p < 0.001 |

TABLE 3
(Caucasian, >50 years old):

| | Active | | | Placebo | | | Unpaired |
|---|---|---|---|---|---|---|---|
| | Mean +/− SD | Average | | Mean +/− SD | Average | | t test |
| | (arbitrary | variation | paired t test | (arbitrary | variation | Paired t test | versus |
| | unit) | (%) vs D 0 | versus D 0 | unit) | (%) vs D 0 | versus D 0 | placebo |
| D 0 | 8850 ± 3438 | | | 7987 ± 3984 | | | |
| D 15 | 7645 ± 3532 | −13.6% | P < 0.001 *** | 8284 ± 3886 | +3.7% | ns | ** p < 0.01 |
| D 28 | 7195 ± 3397 | −18.7% | P < 0.001 *** | 8680 ± 4074 | +8.7% | ns | ** p < 0.01 |
| D 56 | 6877 ± 3542 | −22.3% | P < 0.001 *** | 8445 ± 4534 | +5.7% | ns | *** p < 0.001 |

TABLE 4
(Asian):

| | Active | | | Placebo | | | Unpaired |
|---|---|---|---|---|---|---|---|
| | Mean +/− SD | Average | | Mean +/− SD | Average | | t test |
| | (arbitrary | variation | Paired t test | (arbitrary | variation | Paired t test | versus |
| | unit) | (%) vs D 0 | versus D 0 | unit) | (%) vs D 0 | versus D 0 | placebo |
| D 0 | 9086 ± 4230 | | | 7702 ± 3031 | | | |
| D 7 | 6344 ± 3062 | −30.2% | P < 0.01 ** | 6803 ± 3273 | −11.7% | P < 0.05 * | P < 0.05 * |

1. A cosmetic active agent comprising:
(i) a mixture of mannose-6-phosphate and mannose, wherein the molar ratio of mannose-6-phosphate to mannose is from 3:1 to 0.3:1;
(ii) copper ions;
(iii) a first amino acid selected from the group consisting of lysine, arginine, histidine, and mixtures thereof;
(iv) a second amino amino acid selected from the group consisting of proline, aspartic acid, glutamic acid, and mixtures thereof; and
(v) an acid selected from the group consisting of lactic acid, malic acid, succinic acid, fumaric acid, maleic acid, pyruvic acid, citric acid, gluconic acid, lactobionic acid, sorbic acid, tartaric acid, oxalic acid, 2-pyrrolidone-5-carboxylic acid and mixtures thereof.

2. The cosmetic active agent according to claim 1, wherein the molar ratio of mannose-6-phosphate to mannose is from 2:1 to 1:1.

3. The cosmetic active agent according to claim 1, comprising mannose-6-phosphate in a concentration of 30 to 220 mM.

4. The cosmetic active agent according to claim 1, comprising from 0.5 to 5.0 wt % of mannose.

5. The cosmetic active agent according to claim 1, wherein the copper ions are provided as a copper salt selected from the group consisting of copper sulphate, copper phosphate, copper carbonate, copper chloride, copper acetate, copper malate, copper succinate, copper fumarate, copper maleate, copper pyruvate, copper citrate, copper gluconate, copper glucuronate, copper lactobionate, copper sorbate, copper tartrate, copper oxalate, copper lactate, copper pyroglutamate, copper prolinate, copper aspartate, copper glutamate, and mixtures thereof.

6. The cosmetic active agent according to claim 1, comprising copper ions in a concentration of about 10mM to about 40 mM.

7. The cosmetic active agent according to claim 1, wherein the first amino acid comprises or consists of lysine and/or the second amino acid comprises or consists of proline.

8. The cosmetic active agent according to claim 1, comprising the first amino acid and the second amino acid in a molar ratio of about 3:5 to about 5:2.

9. The cosmetic active agent according to claim 1, comprising the acid in an amount for the cosmetic active agent to have a pH of about 3.8 to about 6.0.

10. The cosmetic active agent according to claim 1, further comprising another amino acid or salt or ester thereof, wherein the amino acid is selected from the group consisting of glutamine, asparagine, glycine, hydroxyproline, serine, methionine, threonine, and mixtures thereof.

11. The cosmetic active agent according to claim 1, further comprising an additive selected from the group consisting of sodium hydroxide, 1,2-propanediol, 1,3-propanediol, 1,3-butanediol, 2,3-butanediol, 1,4-butanediol, glycerol, sodium phosphate and mixtures thereof.

12. The cosmetic active agent according to claim 1, comprising:

| | |
|---|---|
| 2.5-3.5 wt % | mannose-6-phosphate sodium salt |
| 0.7-1.7 wt % | mannose |
| 0.7-1.1 wt % | lysine |
| 1.0-1.4 wt % | proline |
| 0.2-0.4 wt % | copper sulphate |
| 0.8-1.5 wt % | lactic acid |

13. A Cosmetic composition comprising the cosmetic active agent according to claim 1 and a cosmetically acceptable excipient.

14. A method of reducing the number and/or size of pores in human skin, comprising the step of applying the cosmetic active agent according to claim 1 to the human skin.

15. A cosmetic method of reducing parakeratosis in human skin, comprising the step of applying the cosmetic active agent according to claim 1 to the human skin.

16. A cosmetic method of restoring the epidermal integrity of human skin, comprising the step of applying the cosmetic active agent according to claim 1 to the human skin.

17. A method of boosting collagen synthesis in human skin, comprising the step of applying the cosmetic active agent according to claim 1 to the human skin.
